(19) United States
(12) Patent Application Publication
(10) Pub. No.: US 2015/0064715 A1
(43) Pub. Date: Mar. 5, 2015

(54) URINARY BIOMARKERS OF RENAL AND MITOCHONDRIAL DYSFUNCTION

(71) Applicant: MUSC FOUNDATION FOR RESEARCH DEVELOPMENT, Charleston, SC (US)

(72) Inventors: Rick SCHNELLMANN, Mount Pleasant, SC (US); John ARTHUR, Mount Pleasant, SC (US); Craig BEESON, Charleston, SC (US)

(73) Assignee: MUSC FOUNDATION FOR RESEARCH DEVELOPMENT, Charleston, SC (US)

(21) Appl. No.: 14/471,696

(22) Filed: Aug. 28, 2014

Related U.S. Application Data
(60) Provisional application No. 61/870,937, filed on Aug. 28, 2013.

(51) Int. Cl. C12Q 1/34; C07K 16/18; C12N 9/14

(52) U.S. Cl. CPC C12Q 1/34; C12N 9/14; C07K 16/18; G01N 2333/914; G01N 2800/347; G01N 2800/04; C12Y 306/03014
USPC 435/7.4; 435/18; 530/387.9; 530/391.1

(57) ABSTRACT

The present invention provides methods of detecting mitochondrial dysfunction or acute kidney injury (AKI) by measuring the urinary protein levels of the ATP synthase (ATPS) beta subunit or cleavage products thereof in a subject.

This application claims benefit of priority to U.S. Provisional Application Ser. No. 61/870,937, filed Aug. 28, 2013, the entire contents of which are hereby incorporated by reference.

The invention was made with government support under Grant Nos. R01 DK080234 and ES023767-01 awarded by the National Institutes of Health. The government has certain rights in the invention.

### INCORPORATION OF SEQUENCE LISTING

The sequence listing that is contained in the file named “MESC.P0077US_ST25.txt”, which is 12 KB (as measured in Microsoft Windows®) and was created on Aug. 28, 2014, is filed herewith by electronic submission and is incorporated by reference herein.

### BACKGROUND OF THE INVENTION

1. Field of the Invention

The present invention relates generally to the field of nephrology. More particularly, it concerns a method of detecting mitochondrial dysfunction, acute kidney injury, and other types of renal dysfunction in a subject.

2. Description of Related Art

Diverse acute insults from surgery, trauma, ischemia/reperfusion (I/R), and drug toxicity lead to mitochondrial dysfunction and result in cell injury and death in many organs/tissues (e.g., heart, lung, brain, liver and kidney). Furthermore, mitochondrial dysfunction can also contribute to cell injury through increased production of reactive oxygen and nitrogen species. Mitochondrial dysfunction is also a component of many chronic diseases, such as metabolic syndrome, diabetes, neurodegenerative diseases, and aging. Acute kidney injury (AKI) is common among hospitalized patients and the incidence of AKI is increasing. Morbidity and mortality are higher in patients with renal dysfunction and the mortality rate rises as the dysfunction gets worse (Hoste et al., 2006). AKI is also associated with increases in ICU days, hospital days, and discharge to extended care facilities (Mangano et al., 1998). While the importance of mitochondrial dysfunction in AKI in animals has been documented, similar data in humans are minimal because renal tissue for mitochondrial analysis is not normally available (Hall and Unwin, 2007). Thus, there is a great need for non-invasive biomarkers of renal mitochondrial dysfunction.

### SUMMARY OF THE INVENTION

In one embodiment, the present disclosure provides a method of detecting mitochondrial dysfunction in a subject, such as a subject having acute kidney injury (AKI), comprising measuring an elevated level of an adenosine triphosphate synthase beta (ATPSβ, e.g., SEQ ID NO: 2; as known as ATP5B, NCBI accession no. NP_{—}001677, incorporated herein by reference) protein in a sample from said subject. In a further aspect, a method for detecting mitochondrial dysfunction comprises detecting the presence of an ATPSβ cleavage product in a sample from a subject. For example, the ATPSβ cleavage product can be a peptide fragment such as the fragment provided as SEQ ID NO:1, 4, 5, 6, 7, 8 or 9 or a ˜20-25 kDa fragment from the C-terminus of ATPSβ. In some preferred aspects a sample from the subject is a urine sample.

Thus, in a further embodiment there is provided an assay method comprising selectively measuring a level of ATPSβ or an ATPSβ cleavage product in a biological sample from a mammalian subject. In some aspects, the subject has or is suspected of having a mitochondrial dysfunction or is being treated for mitochondrial dysfunction (e.g., a subject being treated with formoterol). In still further aspects, an assay of the embodiments can be used to monitor recovery from mitochondrial dysfunction in a subject. In other aspects, the subject has or is suspected of having kidney dysfunction. In further aspects, the kidney dysfunction is a kidney injury (e.g., AKI), chronic kidney disease or diabetic nephropathy. In certain aspects, the subject has undergone cardiac surgery. For example, the subject may have been put on bypass during the cardiac surgery. In certain aspects, the subject may have ischemia/reperfusion, sepsis, or been exposed to drugs, toxicants, contrast agents, or other insults. In still further aspects, the subject has diabetes or metabolic disease. In preferred aspects, the subject is a human. In certain aspects, the ATPSβ protein levels may be measured before and after the renal injury. In this aspect, the change in levels before and after renal injury may be used to predict renal recovery. In further aspects, the AKI may be stage 1, 2, or 3 AKI.

Samples for use accordingly to the embodiments may be any biological sample, such a blood, saliva, urine, stool or solid tissue sample. In preferred aspects, the sample is a urine sample. In some aspects, detecting or measuring the level of an ATPSβ or an ATPSβ cleavage product comprises performing an immunological assay or performing mass spectroscopy (e.g., comprising multiple reaction monitoring). In some aspects, the immunological assay may be a Western blot, a dot blot, an antibody array, a capillary immune-detection method, isoelectric focusing, an immune precipitation method, immunohistochemistry or an ELISA assay. In further aspects, measuring the level of an ATPSβ or an ATPSβ cleavage product comprises normalizing the measured level to a reference. In some cases, the reference may be the total protein level in the sample, the level of another polypeptide in the sample, or the level of creatinine in the sample.

In still further aspects a method of the embodiments additionally comprises selectively measuring a level of at least one other protein in the sample. For example, the at least one other protein may be ATPS alpha, ATPS gamma, ATPS delta, IL-18, IL-6, VEGF, MCP-1, IL-lra, IL-8, GRO alpha, LIF, IL-10, Eotaxin, VCAM-1, RANTES, TNF-alpha, MIP-1 alpha, Renin, NGAL, KIM-1, L-FABP, HGF, Netrin-1, Clusterin, Fetuin-A, Cystatin-C, Albumin, Beta-2-microglobulin, RBP, Alpha-1 antitrypsin, 8-Isoprostane, TFF-3, NAG, and/or TRAIL. Thus, in one aspect, a method may comprise measuring an elevated level of at least one of IL-18, Interleukin 18; IL-6, Interleukin 6; VEGF, Vascular endothelial growth factor; MCP-1, Monocyte chemotactic protein-1; IL-lra, Interleukin 1 receptor antagonist; IL-8, Interleukin 8; GRO alpha, Growth related oncogene alpha; LIF, Leukemia inhibitory factor; IL-10, Interleukin 10; VCAM-1, Vascular cell adhesion molecule-1; RANTES, Regulated on activation, normal T cell expressed and secreted; TNF-alpha, Tumor necrosis factor alpha; MIP-1 alpha, Macrophage inflammatory protein-1alpha; NGAL, Neutrophil gelatinase associated lipocalin; KIM-1, Kidney injury molecule-1; L-FABP, Liver type fatty acid binding protein, HGF, Hepatocyte growth factor; RBP, Retinol binding protein; TFF-3, Trefoil factor 3; NAG, N-acetyl-beta-D-glucosaminidase; TRAIL, TNF-related apoptosis-inducing ligand in a urine sample from a subject. In this aspect, the method may comprise calculating a ratio of ATPSβ protein (and/or ATPSβ cleavage product) and at least one of the additional markers listed above. In this case, it may be the ratio of ATPSβ protein to a secondary marker (i.e. a relative level of ATPSβ protein) that is indicative of mitochondrial dysfunction. In some aspects, the assay further comprises measuring the level of creatinine in the sample. In a further aspect, a method may comprise measuring the level of ATPSβ protein or detecting an ATPSβ cleavage product in at least one control sample.

In a further embodiment there is provided an immunological complex comprising an ATPSβ cleavage product and an ATPSβ-binding antibody. In some aspects, the ATPSβ cleavage product has a mass of between about 20 and 15 kDa or is a peptide comprising or consisting of the sequence of SEQ ID NO: 1, 4, 5, 6, 7, 8 or 9. In some aspects, the ATPSβ cleavage product is a canine, feline, equine or human ATPSβ cleavage product. In certain aspects, immunological complex (comprising the ATPSβ cleavage product or the ATPSβ-binding antibody) is immobilized on a surface. For example, the surface may be a synthetic or polymeric substrate, which may be a bead, a blot, a slide or a well. In some aspects, the immunological complex further comprises a detectable label, such as a label bound or conjugated to the ATPSβ cleavage product or the ATPSβ-binding antibody. In some aspects, the label is attached to an antibody that binds the ATPSβ cleavage product or the ATPSβ-binding antibody.

In certain aspects, a method may comprise reporting whether the subject has an elevated level of urine ATPSβ (or reporting the presence or level of a ATPSβ cleavage product). In a further aspect, the reporting may comprise providing an oral, written or electronic report. In yet another aspect, the reporting may comprise providing a report to the subject, a healthcare worker or a payee.

In still a further embodiment, an isolated peptide comprising the sequence of SEQ ID NO:1, wherein the peptide is no more than 50, 40, 30 or 20 amino acids in length. In some aspects, the isolated peptide consists of the sequence of SEQ ID NO: 1, 4, 5, 6, 7, 8 or 9. In a related embodiment there is provided an antibody that specifically binds to a peptide comprising or consisting of the sequence of SEQ ID NO: 1, 4, 5, 6, 7, 8 or 9. In some aspects, the recombinant antibody does not bind to an ATPSβ cleavage product lacking the sequence of SEQ ID NO:1, 4, 5, 6, 7, 8 or 9. In certain aspects, the antibody is recombinant, such as a monovalent scFv, a bivalent scFv, or a single domain antibody. In further aspects, the antibody may be an IgG, IgM, IgA, IgE or an antigen binding fragment thereof, such as a Fab′, a F(ab′)2, or a F(ab′)3. In further aspects, the antibody may be a non-human antibody such a mouse or rabbit antibody. In some cases, the antibody is part of a polyclonal antiserum and may be a monoclonal antibody. In still further aspects, the antibody is attached to a detectable label, such a fluorescent label of a report protein or an enzyme.

As used herein the phrase “selectively measuring” refers to methods wherein only a finite number of protein (e.g., urinary protein) markers are measured rather than assaying essentially all proteins in a sample. For example, in some aspects “selectively measuring” protein markers can refer to measuring no more than 100, 75, 50, 25, 15, 10 or 5 different protein markers in a sample.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

As used herein, the term “biological sample” is used in its broadest sense and can refer to a bodily sample obtained from a subject (e.g., a human). For example, the biological sample can include a “clinical sample”, i.e., a sample derived from a subject. Such samples can include, but are not limited to: peripheral bodily fluids, which may or may not contain cells, e.g., blood, urine, plasma, mucous, bile pancreatic juice, supernatant fluid, and serum; tissue or fine needle biopsy samples; and archival samples with known diagnosis, treatment and/or outcome history. Biological samples may also include sections of tissues, such as frozen sections taken for histological purposes. The term “biological sample” can also encompass any material derived by processing the sample. Derived materials can include, but are not limited to, cells (or their progeny) isolated from the biological sample and proteins extracted from the sample. Processing of the biological sample may involve one or more of, filtration, distillation, extraction, concentration, fixation, inactivation of interfering components, addition of reagents, and the like. In certain preferred aspects, a biological sample is a blood or urine sample.

By “subject” or “patient” is meant any single subject for which therapy or diagnostic test is desired. In this case the subjects or patients generally refer to mammalian subjects, such as dogs, cats, horses and, in particular, humans.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C—Serum creatinine (A) and renal mitochondrial protein (B and C) after glycerol-induced AKI in rats over time. Bars represent the average±SEM. Bars with different superscripts are significantly different from one another (p<0.05).

FIG. 2—Serum creatinine after I/R-induced AKI in rats over time. Bars represent the average±SEM. Points with different superscripts are significantly different from one another (p<0.05).

FIG. 3—Relative concentrations of the alpha, beta, delta, gamma, and O subunits of ATP synthase. The relative concentrations of serum albumin, major urinary protein, cystatin-c, fetuin, NGAL, and KIM-1 are also shown.

FIG. 4—Increased urinary ATPSβ in mice subjected to a range of I/R times and grades of kidney injury and unique peptide sequences for urinary ATPSβ in mice. 8-week old male C57BL6 mice weighing 25-30 g were divided into naïve, sham or I/R groups. Mice in I/R group were subjected to bilateral renal pedicle ligation as described previously (see, Funk et al., 2012). Briefly, the renal artery and vein were isolated and blood flow was occluded with a vascular clamp for 5, 10 and 15 min, and mice were euthanized at 24 h after procedure. Blood serum was analyzed for urea nitrogen (BUN) (A); creatinine (B); and urinary neutrophil gelatinase lipocalin 2 (NGAL) (C) as indicators of renal function and/or damage. Urinary ATPSβ was also measured by immunoblot analysis (D). The full-length and cleaved fragments of ATPSβ were quantified by densitometry and normalized to a standard sample and total urinary protein (E,F). Data are expressed as mean±SEM (n=12). * indicates significance from all other groups. (p≦0.05). To validate the immunoblot results, ATPSβ was immunoprecipitated from urine, purified by gel electrophoresis and analyzed by LC-MS/MS analysis. A representative Coomassie gel for immunoprecipitated-urinary ATPSβ detected with mouse monoclonal antibodies is shown (G).

FIG. 5—Disruption of renal mitochondrial ATPSβ protein in mice subjected to a range of I/R times and grades of kidney injury. Representative immunoblots showing renal cortical protein expression of mitochondrial ATPSβ (A) and data quantitated by densitometry and graphed (B) at 24 h after mice subjected to a range of I/R times and grades of kidney injury. Data were normalized by GAPDH, which served as internal control. Data are expressed as mean±SEM (n=6). * Indicated significant relative to sham. (p≦0.05).

FIG. 6—Increased urinary ATPSβ over a time course in mice subjected to I/R-induced AKI. 8-week-old male C57BL/6 mice weighing 25-30 g were subjected to sham surgery or bilateral renal pedicle ligation for 17 minutes. Urine was collected 72 and 144 h following reperfusion. (A) Representative immunoblots showing protein expression of urinary ATPSβ full-length and cleaved fragment in sham or I/R mice. The ATPSβ full-length and cleaved fragments were quantified by densitometry and normalized to a standard sample and total urinary protein. Data are expressed as mean±SEM (n=5-10). * Indicates significance from all other groups. (p≦0.05).

FIG. 7—Urinary ATPSβ in human patients with AKI after cardiac surgery and unique peptide sequences for urinary ATPSβ. Representative immunoblots for urinary full-length and cleaved ATPSβ protein expressions (A); urinary full-length and cleaved ATPSβ normalized to total urinary protein load (B, C); serum creatinine in no AKI and AKI patients 1.5 days after cardiac surgery (D). Data are expressed as mean±SEM (n=16). * Significant from No AKI. (p≦0.05). (E) Mass spectrum from urine of a human with AKI after cardiac surgery showing the MS/MS fragmentation pattern of the tryptic peptide VVDLLAPYAK (SEQ ID NO: 1) unique for human mitochondrial ATPSβ.

FIG. 8—Panels show H&E stained kidney sections from mice subjected to either sham or a range of I/R times and grades of kidney injuries.

FIG. 9—Results of studies in a rat model of diabetic nephropathy/chronic kidney disease. The levels of urinary and tissue ATPSβ were measured after streptozotocin treatment. Results show that urinary full length and cleaved ATPSβ are elevated three weeks after streptozotocin treatment. Renal tissue ATPSβ was unchanged at four weeks post treatment.

FIG. 10—Results of studies in a mouse model (db/db) of chronic kidney disease. The levels of urinary and tissue ATPSβ were measured in db/db animals and control animals after nine weeks. Results show that urinary full length and cleaved ATPSβ are elevated and renal tissue ATPSβ was decreased.

FIG. 11—Results of studies in a mouse model for renal recovery from ischemia (see FIG. 4 above for methods). Graphs show that formoterol treatment reduces full length and cleaved urinary ATPSβ levels and improved renal function. Male C57BL/6 were subjected to 20 min of ischemia followed by reperfusion. Mice were treated daily starting at 24 h after I/R with vehicle or formoterol. Full length (A) and cleaved (B) urinary ATPSβ levels were measured by immunoblot and quantified by densitometry.

FIG. 12A-B—(A) Shows the sequence of rat ATPSβ (SEQ ID NO: 3; NCBI accession no. NP_{—}599191). Underlined amino acids represent the peptide cleavage products that were identified in the mass spectrum (from N-terminus to C-terminus SEQ ID NOs: 4, 5, 1, 6, 7, 8 and 19). (B) Shows a sequence alignment between human ATPSβ (SEQ ID NO: 2) and rat ATPSβ (SEQ ID NO: 3) using Clustal Omega. Overall sequence identity between the two polypeptides is 97%, however, the identified peptide cleavage products share 100% identity.

### DESCRIPTION OF ILLUSTRATIVE EMBODIMENTS

Diverse acute insults from surgery, trauma, ischemia/reperfusion (I/R), and drug toxicity lead to mitochondrial dysfunction and result in cell injury and death in many organs/tissues (e.g., heart, lung, brain, liver and kidney). Thus, there is a great need for non-invasive biomarkers of mitochondrial dysfunction. At this time, few tools are available to measure early markers of mitochondrial dysfunction in humans and animals resulting from surgery, trauma, drug exposure, and disease processes. Current biomarkers of organ dysfunction do not focus on mitochondrial dysfunction and markers of mitochondrial dysfunction are limited to invasive muscle biopsies, organ ATP measurements, or functional measurements in isolated mitochondria. NMR and mass spectrometry (MS)-based metabolic profiling and metabonomic approaches have been explored to measure whole-body changes in metabolic functions; however, data from these approaches only summarize metabolic changes throughout the body and do not indicate organ-specific effects (Coen et al., 2008). Consequently, new non-invasive assays are needed that specifically focus on mitochondrial dysfunction within the kidney. The most useful biomarkers for mitochondrial disease or dysfunction will be those that are easily measured over long periods of time, are non-invasive, and correlate with acute and chronic mitochondrial dysfunction.

The inventors show that urinary protein levels of mitochondrial ATP synthase (ATPS) subunits are sensitive and specific markers of mitochondrial dysfunction in AKI in animals and humans. In particular, urinary ATPSβ (and its cleavage products) increase in humans with AKI following cardiac surgery, compared to control humans with normal renal function and humans with no AKI following cardiac surgery. Complementing the human studies, urinary ATPSβ levels increased in rats subjected to ischemia/reperfusion (I/R)- and glycerol-induced AKI when renal mitochondrial dysfunction was present. In models of diabetic nephropathy/chronic kidney disease, urinary ATPSβ levels increased in both rat and mouse models. These studies represent new urinary markers of renal mitochondrial dysfunction in humans and animals. Finally, these biomarkers can be readily translated into laboratory and clinical practice.

### I. BIOMARKER DETECTION

The expression of biomarkers such as ATPSβ protein (or its cleavage products) may be measured by a variety of techniques that are well known in the art. For example, measuring a level of a protein may comprise performing immunohistochemistry, an ELISA (e.g., a sandwich ELISA), a radioimmunoassay (RIA), an immunoradiometric assay, a fluoroimmunoassay, a chemiluminescent assay, a bioluminescent assay, a gel electrophoresis, a Western blot analysis, a mass spectrometry analysis, a protein microarray, a capillary protein immune detection system.

In some aspects, a marker level (such as a ATPSβ protein level) may be compared to the level of a control marker or with the corresponding marker from a control, sample. For example, in some cases the control maker is a biomarker (e.g., a protein) that displays consistent stable levels regardless of mitochondria dysfunction. Likewise, in some aspects a marker level is assessed in control sample, such as a sample from a subject known to have (or that does not have) mitochondria dysfunction.

Control marker levels or marker levels from a control sample may be determined at the same time as a test sample (e.g., in the same experiment) or may be a stored value or set of values, e.g., stored on a computer, or on computer-readable media. If the latter is used, new sample data for the selected marker(s), obtained from initial or follow-up samples, can be compared to the stored data for the same marker(s) without the need for additional control experiments.

A. Methods of Protein Detection

In some aspects, measuring the expression of said genes comprises measuring protein expression levels. Measuring protein expression levels may comprise, for example, performing an ELISA, Western blot, immunohistochemistry, or binding to an antibody array. In certain aspects, determining a level of ATPSβ protein in a sample comprises contacting the sample with an antibody to that binds to ATPSβ protein or an ATPSβ cleavage product.

An enzyme-linked immunosorbent assay, or ELISA, may be used to measure the differential expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art. Single- and Multi-probe kits are available from commercial suppliers, e.g., Meso Scale Discovery (MSD). These kits include the kits referenced in the Examples hereto.

An antibody microarray may also be used to measure the differential expression (and/or differential cleavage) of a plurality of protein biomarkers. For this, a plurality of antibodies is arrayed and covalently attached to the surface of the microarray or biochip. A protein extract containing the biomarker proteins of interest is generally labeled with a fluorescent dye or biotin. The labeled biomarker proteins are incubated with the antibody microarray. After washes to remove the unbound proteins, the microarray is scanned. The raw fluorescent intensity data may be converted into expression values using means known in the art.

### II. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Histologic and Mitochondrial Function Data in Animal Models of AKI

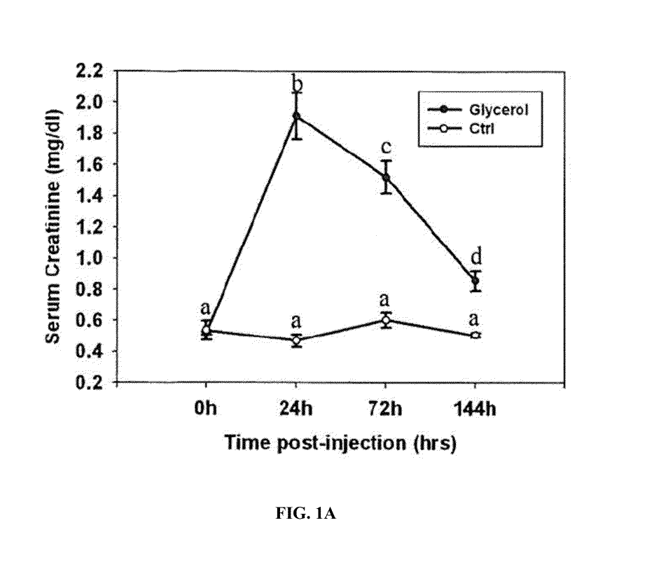

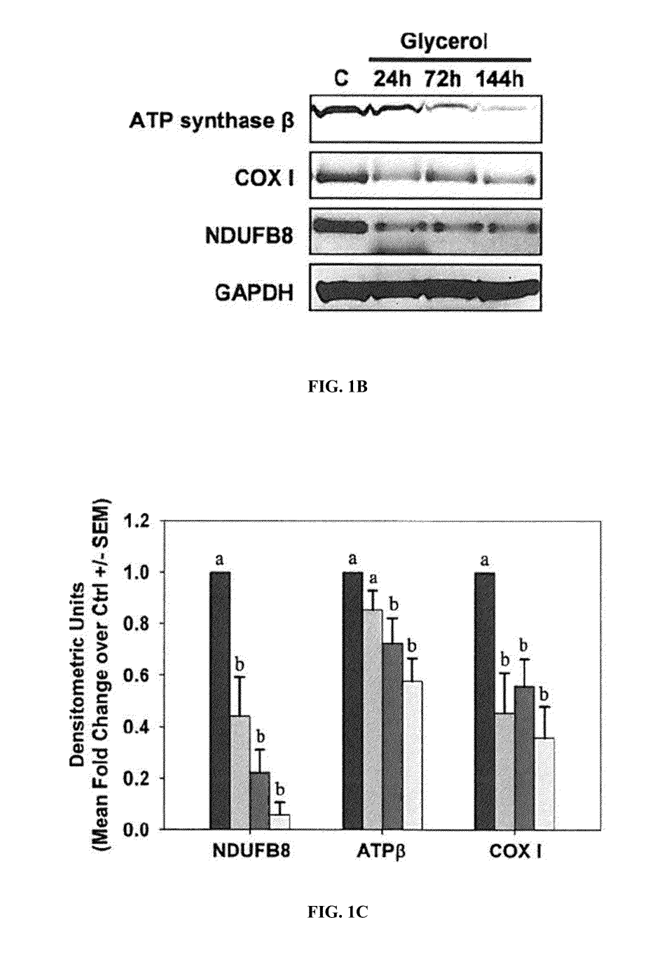

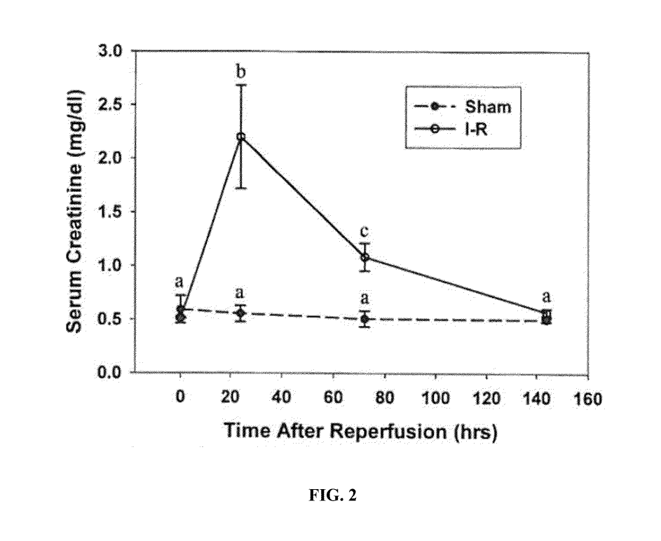

The inventors used a rat model of myoglobinuric-AKI (glycerol), an established model of “crush injury” AKI (Kim et al., 2010; Zager, 1996; Zager et al., 1991). In this model, glycerol (10 mL/kg) is injected into each hind limb in two equally divided doses. Various times after initiation, urine and serum were collected and renal tissue harvested for immunoblot analysis. Serum creatinine was maximal 24 h after glycerol injection and decreased over 144 h, illustrating renal dysfunction and partial recovery (FIG. 1A). Immunoblot analysis of renal tissue lysates revealed time dependent loss of three mitochondrial proteins (NDUFB8, COX1, ATPSβ), demonstrating mitochondrial dysfunction in the kidney (FIGS. 1B and 1C). The inventors also used a rat model of renal FR-induced AKI (Zhuang et al., 2009; Leonard et al., 2008). Serum creatinine was maximal 24 h after I/R and decreased over 144 h (FIG. 2).

The inventors analyzed urine from three rats with glycerol-induced AKI and three control rats. Sample preparation, chromatography, and mass spec analysis were done as described above for the human samples. Spectra were analyzed using Mascot suing the SwissProt database (selected for Rattus, 7568 entries). Mascot was searched with a fragment ion mass tolerance of 0.50 Da and a parent ion tolerance of 10.0 ppm. Scaffold was used to validate MS/MS based peptide and protein identifications. Identifications required that ion scored must be greater than both the associated identity scores and 30. Protein identifications were accepted if they contained at least two identified peptides. False discovery rate calculated in Scaffold using the reverse rat SwissProt database was 0.0%. Quantification was performed in Scaffold using the quantitative value parameter, which is based on spectral counting.

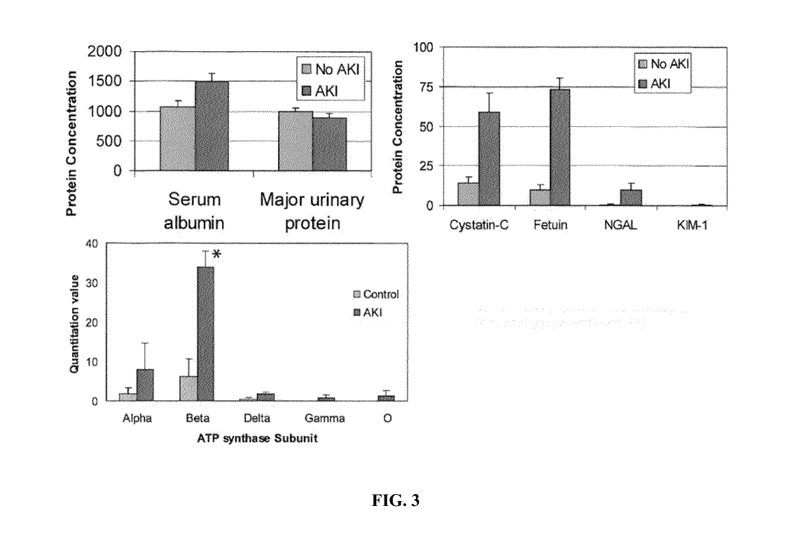

Two hundred fifty-nine proteins were identified with very high confidence, and 114 proteins were nominally more abundant in the control group; 143 were nominally more abundant in the AKI group and two proteins were nominally identical. Serum albumin and major urinary protein were the most abundant proteins and were not different between groups (FIG. 3). Several known biomarkers of kidney injury were identified in the urine from the proteomic data. The concentrations of cystatin C and fetuin were statistically greater in the urine from the AKI animals. The concentrations of NGAL and KIM-1 were nominally larger in the AKI animals but the difference did not reach statistical significance in the relatively small group.

The inventors also compared the abundance of the mitochondrial proteins seen in the urine sample. Forty-seven of the 259 proteins that were identified are associated with mitochondria. The inventors identified four of the five subunits of the mitochondrial ATPS catalytic core (alpha, beta, delta, and gamma) as well as ATPS O, which is in the delta family. All five subunits had nominally higher abundance in the AKI rats although only the beta subunit was significantly different between groups (FIG. 3). Two other mitochondrial proteins were significantly increased in the AKI group (alanine-glyoxylate aminotransferase 2 and dihydrolipolylysine-residue succinyl-transferase component of 2-oxoglutarate dehydrogenase complex). Other mitochondrial proteins showed nominal decreases or increases in the mean abundance, which did not reach statistical significance in this small group.

### Example 2

### Studies in Animal Models of Diabetic Nephropathy and Chronic Kidney Disease

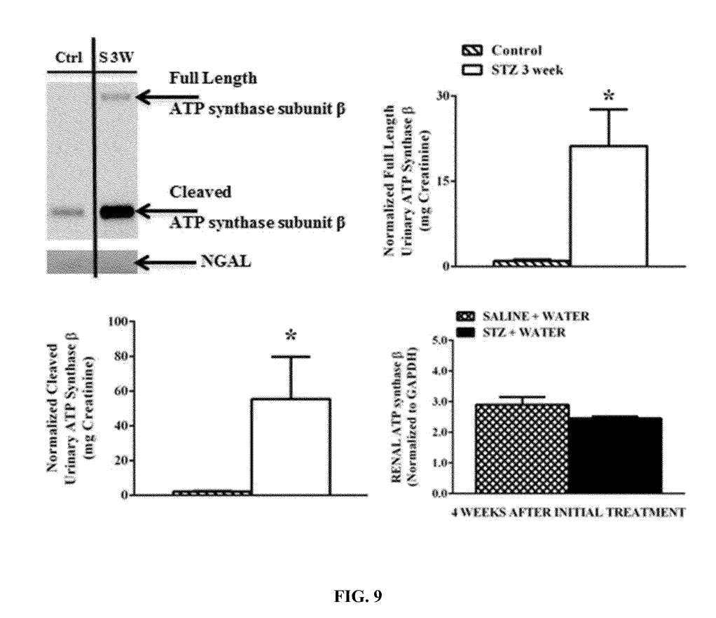

The levels of urinary ATPSβ and ATPSβ cleavage products were measured in rats following induction of diabetes by administration of streptozotocin (to kill pancreatic beta cells). As shown in FIG. 9, three weeks after streptozotocin administration significant elevated levels of both ATPSβ and ATPSβ cleavage product were detectable.

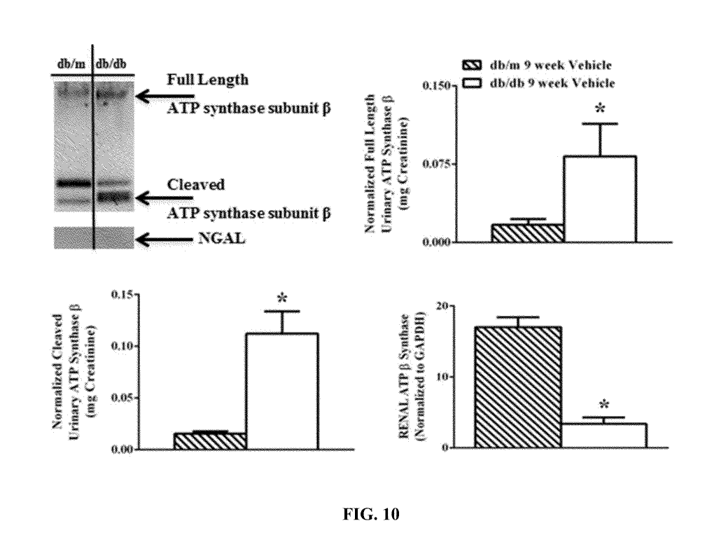

The role of urinary ATPSβ as a marker in chronic kidney disease was also assessed using the db/db mouse model system (see, Sharma et al., 2003). As shown in FIG. 10, db/db mice showed significantly increased levels of urinary full length and cleaved ATPSβ, whereas renal levels of ATPSβ decreased as compared to heterozygous db/m animals.

These studies further demonstrate the broader effectiveness of ATPSβ as a marker for mitochondria dysfunction in disease. In particular, elevated levels of urinary ATPSβ and ATPSβ cleavage products were shown to correlate with the onset of both diabetic nephropathy and chronic kidney disease.

### Example 3

### ATPSβ can be Used to Monitor Recovery for Mitochondrial Dysfunction

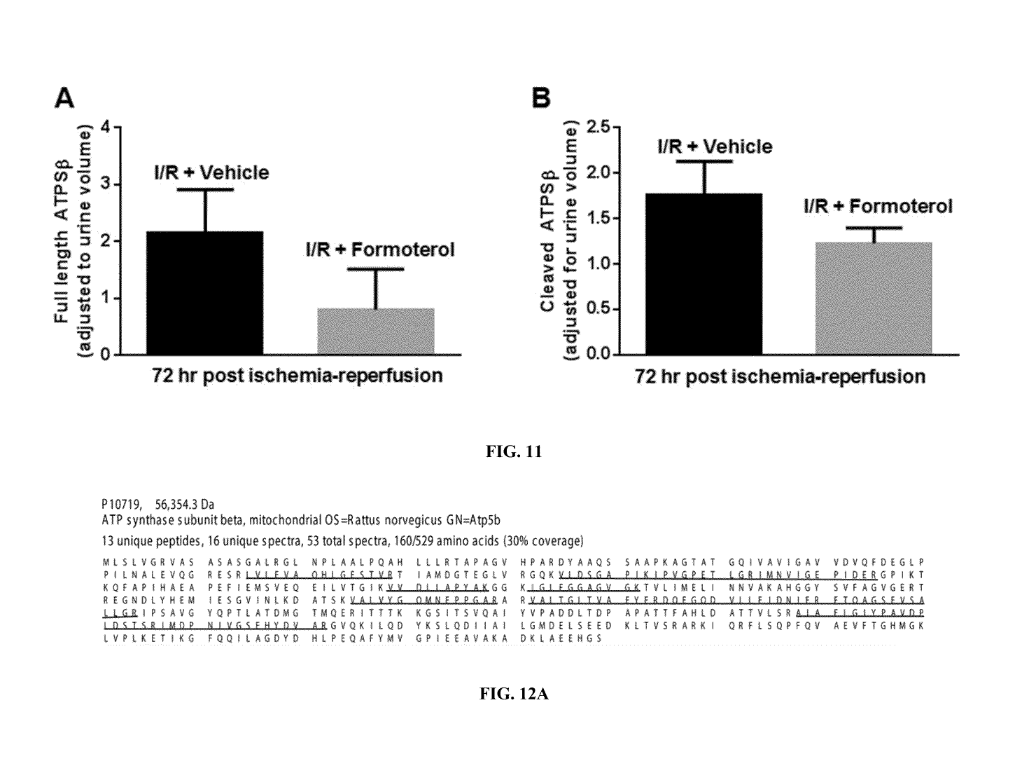

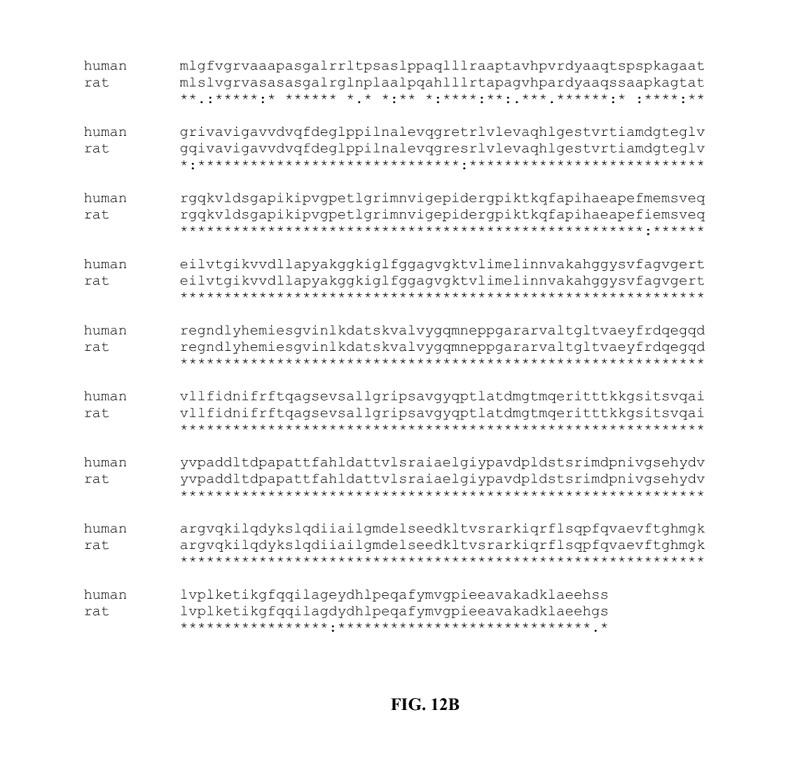

Formoterol has been demonstrated to restore mitochondrial function following ischemia/reperfusion induced AKI (Jesinkey et al., 2014, incorporated herein by reference). To determine if ATPSβ levels could be used to monitor recovery in treated animals, male C57BL/6 were subjected to 20 min of ischemia followed by reperfusion. Mice were treated daily starting at 24 h after I/R with vehicle or formoterol. Urinary ATPSβ levels were then tested in treated or control animals. As shown in FIG. 11, full length (A) and cleaved (B) urinary ATPSβ levels were both decreased in the formoterol-treated animals indicating that ATPSβ level can be used to monitor recovery from mitochondria dysfunction.

### Example 4

### Studies in Humans and Mouse Models of AKI

### Materials and Methods

Animals and Treatments

Eight-week-old male C57BL6 mice weighing 25-30 g were divided into naïve, sham or I/R groups. Mice in I/R group were subjected to bilateral renal pedicle ligation as described previously (Funk et al., 2012). Briefly, renal artery and vein were isolated and blood flow was occluded with a vascular clamp for 5, 10 and 15 min, and mice were euthanized at 24 h after procedure, at which time serum and urine were collected from all mice and kidneys harvested for further analysis. A separate 15 min ischemia experiment was conducted as described above and mice were euthanized at 72 and 144 h after procedure, at which time serum and urine were collected from all mice and kidneys harvested for further analysis. Appropriate sham controls were maintained in all the above-mentioned experiments. All procedures involving animals were performed with approval from the IACUC in accordance with the NIH Guide for the Care and Use of Laboratory Animals.

Human Urine Samples

A subset of human urine samples from a recently published study were used in further studies. All of the details of sample collection, processing, informed content and inclusion/exclusion criteria are provided in Alge et al., 2013, incorporated herein by reference. Urine samples were obtained as part of NIDDK-funded multicenter trial (NIH #DK080234) to identify prognostic markers in urine after cardiac surgery. Urine was collected from cardiac surgery patients at Duke University, George Washington University, MUSC, and Chattanooga, Tenn. who develop AKI and those who do not. Briefly, urine was collected using a standard operating procedure from patients who had cardiac surgery. Protease inhibitors were added to each sample, supernatant collected after centrifugation at 1000×g, and aliquots frozen at −80° C. Samples were shipped on dry ice where they are kept frozen until needed. Corresponding clinical data including demographics, baseline, collection and maximum values for serum creatinine, electrolytes, type of surgery, cardiopulmonary bypass time, preexisting diseases, dialysis status, days to discharge and mortality status were collected (Table 1). Samples were also collected from patients who had cardiac surgery but did not develop AKI. These samples have been collected by the MUSC CTSA biobank and are linked to medical record numbers so that demographic and clinical information regarding these subjects can be obtained.

Chemicals

Unless stated otherwise, all chemicals and biochemicals were purchased from Sigma Chemical Co. (St. Louis, Mo.). Rabbit polyclonal anti-neutrophil gelatinase-associated lipocalin (NGAL) and mouse monoclonal anti-ATPSβ were purchased from Abcam Inc. (Cambridge, Mass.); and the loading control glyceraldehyde 3-phosphate dehydrogenase (GAPDH) was obtained from Fitzgerald International Inc. (Acton, Mass.). Anti-rabbit and anti-mouse secondary antibodies conjugated with horseradish peroxidase were obtained from Pierce (Rockford, Ill.). All LC-MS/MS reagents were LC-grade pure and purchased from Waters (Milford, Mass.). Protein-A agarose beads used in immunoprecipitation protocol were purchased from Roche (Indianapolis, Ind.).

Assessment of Renal Function and Damage

Mice were placed in metabolic cages (Tecniplast, Philadelphia, Pa.) for 24 h urine collections. Renal function was monitored by measuring 24 h urine volume, serum/urine creatinine and serum blood urea nitrogen using assay kits (BioAssay Systems, Hayward, Calif.) as per manufacturer's instructions. Urinary NGAL was measured by immunoblot analysis and normalized to a common sample that was included in all the gels. Renal tissues were fixed in 4.5% buffered formalin, dehydrated, and embedded in paraffin. For general histopathology, sections were stained with hematoxylin/eosin.

Renal and Urinary Immunoblot Analysis

Immunoblot analysis using mouse kidney cortex tissue was performed as previously described (see, Korrapati et al., 2012 and Korrapati et al., 2013, each of which is incorporated herein by reference). Urine samples from mice and humans were collected on ice, protease inhibitors added and centrifuged for 10 min at 1000 g. Aliquots were snap frozen and stored at −80° C. Samples were homogenized in 1 volume of protein lysis buffer (1% Triton X-100, 150 mM NaCl, 10 mM Tris-HCl, pH 7.4; 1 mM EDTA; 1 mM EGTA; 2 mM sodium orthovanadate; 0.2 mM phenylmethylsulfonyl fluoride; 1 mM HEPES, pH 7.6; 1 μg/ml leupeptin; and 1 μg/ml aprotinin) using a Polytron homogenizer. The homogenate was stored on ice for 10 min and then centrifuged at 1000 g for 2 min at 4° C. The supernatant was collected; total urinary protein was determined using a bicinchoninic acid kit (Sigma-Aldrich) with bovine serum albumin as the standard. Equal amounts of protein (10 μg) were separated on 4 to 20% gradient SDS-polyacrylamide gels and transferred to nitrocellulose membranes. Membranes with either renal or urinary proteins were blocked either in 5% dried milk or BSA in TBST (0.1% Tween 20 in 1× Tris-buffered saline) and incubated with 1:1000 dilutions of anti-NGAL, anti-ATPSβ, and anti-GAPDH overnight at 4° C. After incubation for 2 h at room temperature with secondary antibodies (1:2000) conjugated with horseradish peroxidase, membrane proteins were detected by chemiluminescence. Renal proteins were quantified and normalized with GAPDH. Urinary proteins were quantified and normalized with a common sample that was included in all the gels, and finally adjusted to total urinary protein.

Urinary ATPSβ Identification Using LC-MS/MS Analysis

Frozen urine aliquots from mice and humans were thawed at 37° C. for 10 min and centrifuged for 10 min at 1000 g and 4° C., and total urinary protein and creatinine values were measured. The sample volume used for trypsin digestion and subsequent proteomic analysis was calculated by normalizing total urinary protein to both urine volume and urine creatinine to eliminate biological variability. LC-MS/MS analysis, ATPSβ peptide identification, normalization of spectral counts with internal standard HIV gp160 protein for each sample was done as previously described (see, Korrapatie et al., 2012 and Alge et al., 2013, each of which is incorporated herein by reference).

ATPSβ Immunoprecipitation and Peptide Identification

Immunoprecipitation was performed according to the method described by Abcam Inc (Cambridge, Mass.). Briefly, mouse urine sample from 15 min I/R group was homogenized in lysis buffer with protease inhibitor cocktail, followed by centrifugation to remove cell debris and protein concentration was estimated. Protein complexes were obtained by incubating pre-cleared lysates with 500 μg of total protein concentration and 10 μg mouse monoclonal anti-ATPSβ (Abcam Inc., Cambridge, Mass.) overnight at 4° C. with gentle agitation. These complexes were mixed with protein A-agarose bead slurry (70-100 μl) on ice and incubated overnight at 4° C. under rotary agitation. When the incubation time is over, centrifuge the tubes, were removed, supernatant was washed in lysis buffer three times (each time centrifuging at 4° C. and removing the supernatant). Finally, the last supernatant was removed and 25-50 μl of 2× loading buffer was added, boiled at 95-100° C. for 5 minutes, centrifuged and supernatant was run on SDS-PAGE gel. Gel was stained with Coomassie and the bands were excised for further LC-MS/MS-based peptide identification as previously described (see, Ball et al., 2006, incorporated herein by reference).

Data and Statistical Analysis

Data are expressed as means±SEM for all the experiments. Multiple comparisons of normally distributed data were analyzed by one-way ANOVA, as appropriate, and group means were compared using Student-Newman-Keuls post-hoc test. Single comparisons were analyzed by Student's t-test where appropriate. The criterion for statistical differences was p≦0.05 for all comparisons.

### Results

Exaggerated Renal Dysfunction and Damage in Mice Subjected to 15 min I/R-AKI.

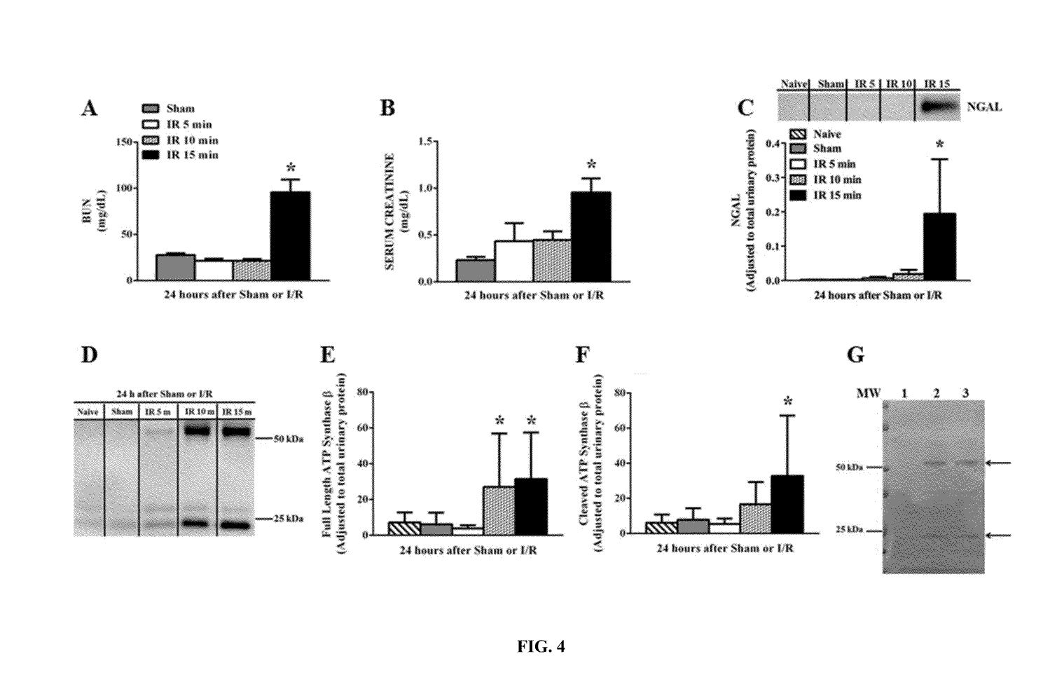

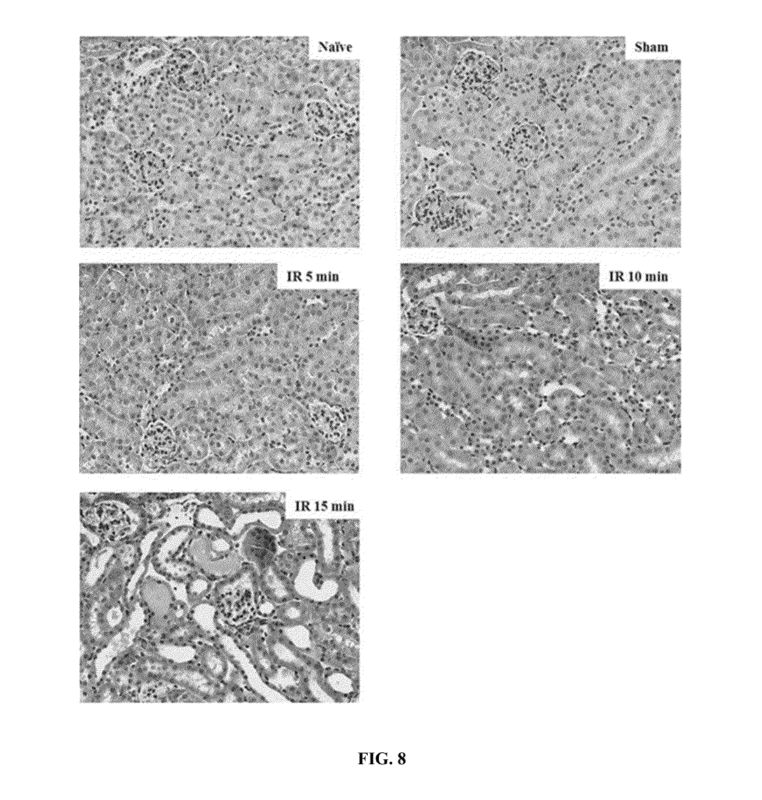

Mice were subjected to sham or I/R by bilaterally ligating renal pedicle for 5, 10 and 15 min and grades of kidney injuries were compared. Blood urea nitrogen (BUN) and serum creatinine were maximal at 24 h in mice subjected to 15 min I/R when compared to mice in sham, 5 and 10 min I/R groups (FIGS. 4A & 4B). Associated with renal dysfunction, mice in 15 min I/R group exhibited extensive proximal tubular necrosis throughout the corticomedullary region characterized by eosinophilic tubules with remnants of karyolytic nuclei when compared to the renal architecture in naïve, sham, 5 and 10 min I/R groups (FIG. 8). There was no histological appearance of renal damage in mice subjected to 5 min ischemia time as when compared to minimal or very mild proximal tubular vacuolization in kidneys of mice in 10 min I/R group (FIG. 8). Urinary NGAL at 24 h after initial procedure in 15 min I/R group was detected when compared to undetectable levels in mice from other groups (FIG. 4C).

Mice Subjected to I/R have Increased Urinary ATP Synthase Subunit β.

For the first time, a full length (˜50 kDa) and a cleaved fragment (˜25 kDa) for urinary ATPSβ protein was identified. Excretion of these two proteins increased 24 h after mice subjected to initial 10 and 15 min I/R procedure when compared to mice in naïve, sham and 5 min I/R groups (FIG. 4D). Both full-length and cleaved urinary ATPSβ were elevated when adjusted to total urinary protein respectively. Full-length ATPSβ showed similar increases in mice subjected to 10 and 15 min I/R when compared to urinary levels in naïve, sham and 5 min I/R mice (FIG. 4E). Whereas, cleaved urinary ATPSβ increased only in 15-min I/R group (FIG. 4F). Immunoprecipitation of ATPSβ in urine samples from a 15 min I/R mouse and analysis by LC-MS/MS confirmed that the unique peptides belonging to mouse mitochondrial ATPSβ could be identified (FIG. 4G).

Mice Subjected to I/R have Increased Renal Cortical Mitochondrial Disruption.

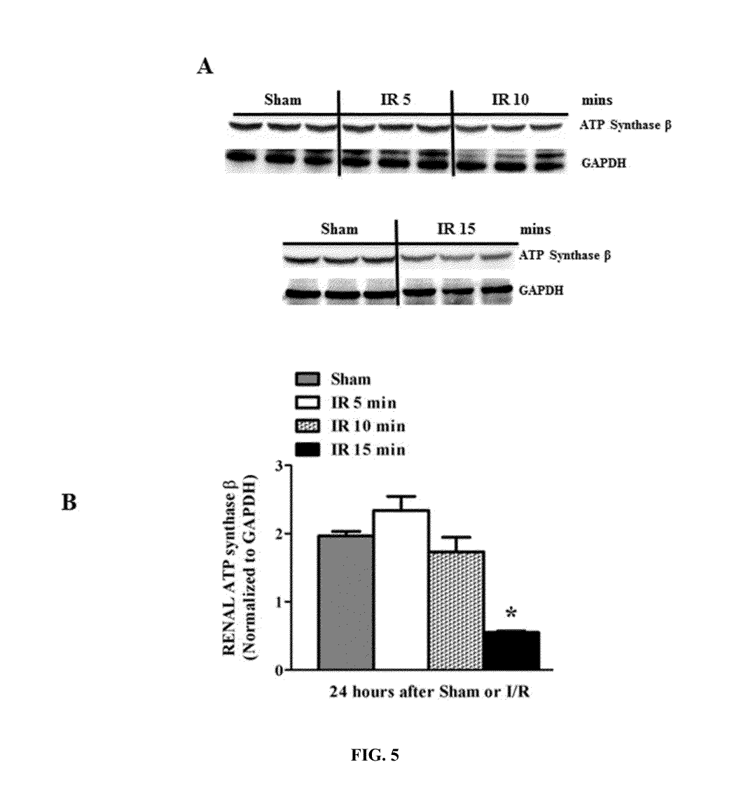

Persistent disruption of renal mitochondrial proteins in mice after I/R has been recently noted (Funk et al. 2012). However, if it was not known if this disruption of renal mitochondrial homeostasis correlates with an increase in urinary ATPSβ. Renal cortical protein expression was analyzed for nuclear-encoded mitochondrial ATPSβ at 24 h after sham or I/R. Interestingly, renal ATPSβ protein decreased in 15 min I/R group alone (FIGS. 5A-B).

Persistent Elevation in Urinary ATPSβ after I/R-AKI and Mitochondrial Disruption Over a Time Course after AKI.

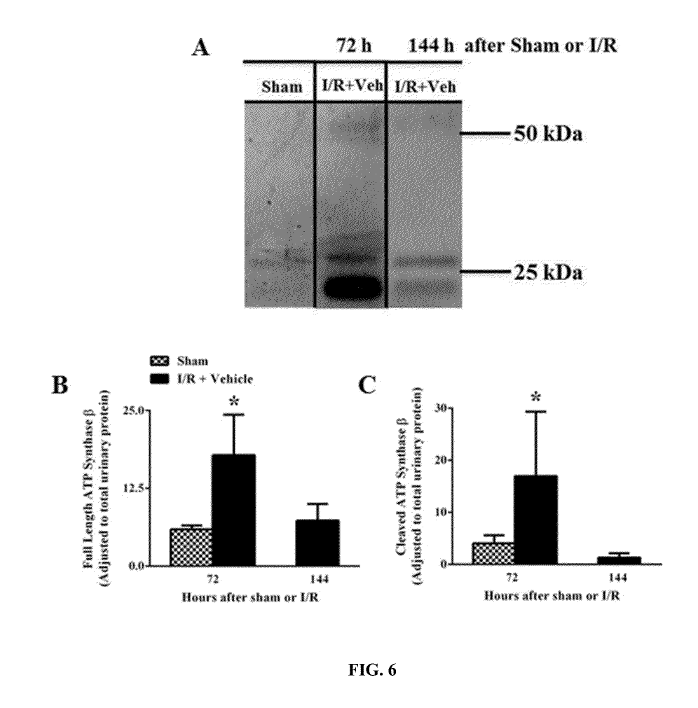

Renal mitochondrial dysfunction in I/R-AKI mice is persistent until 6 days after I/R-AKI with continual suppression of mitochondrial-protein disruption. Accordingly it was tested whether increased urinary ATPSβ levels correlate with persistent renal mitochondrial disruption over a time course. Results of these studies revealed that urinary ATPSβ levels were significantly higher at 72 h after initial procedure in 17 min-I/R mice when compared to mice in sham groups at the same time point (FIG. 6A-C). Data indicate that urinary full-length/cleaved ATPSβ along with their normalized (adjusted to total urinary protein load) values were significantly increased in I/R mice at 72 h when compared to all other groups (FIGS. 6B-C). On the other hand, I/R mice have levels comparable to control mice at 144 h (FIGS. 6A-C).

Patients Who Developed Severe AKI after Cardiac Surgery Had Increased Urinary ATPSβ.

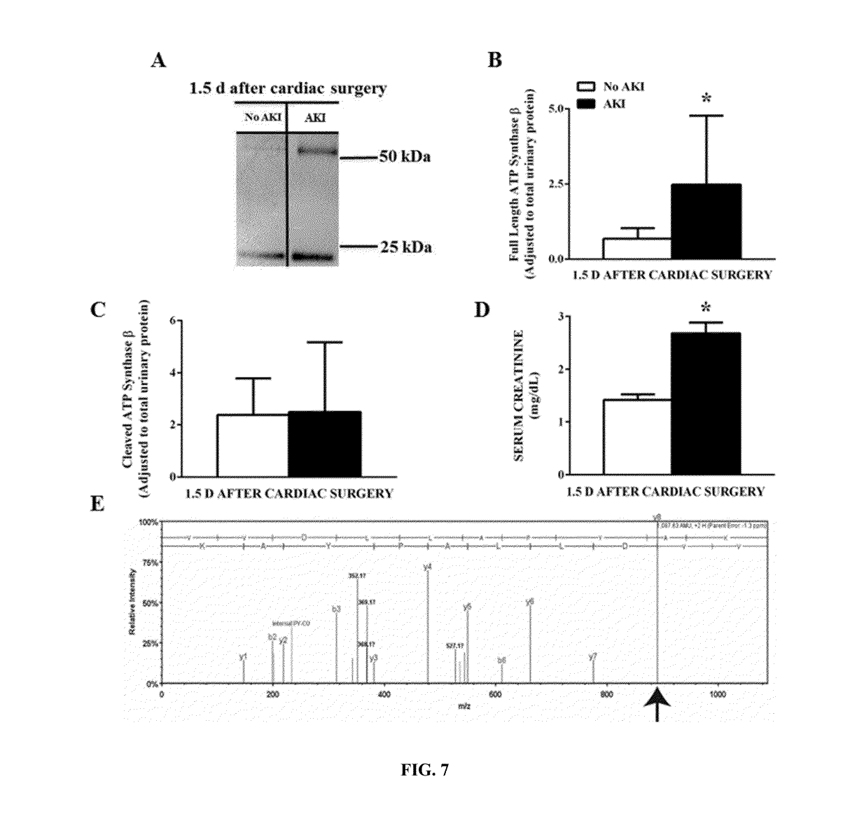

For the first time, a full length (˜50 kDa) and a cleaved fragment (˜25 kDa) for urinary ATPSβ protein have been identified in patients who developed AKI. Excretion of these proteins was increased 1.5 days after cardiac surgery (FIG. 7A) when compared to patients who did not develop AKI after cardiac surgery. When adjusted to total urinary protein, full-length ATPSβ was increased in AKI patients when compared to patients with no AKI (FIG. 4B), while no change was seen in cleaved urinary ATPSβ protein (FIG. 7C). In order to make sure these patients suffered from a severe form of AKI, serum creatinine was analyzed at 1.5 days after cardiac surgery and it was found that AKI patients had a 2-fold increase in serum creatinine (FIG. 7D). Furthermore, LC-MS/MS experiments were performed with urine from patients who developed severe AKI 1.5 days after cardiac surgery and found a unique peptide, VVDLLAPYAK (SEQ ID NO: 1; the same peptide was also identified in FIG. 4G) that is specific to human mitochondrial ATPSβ (FIG. 7E).

TABLE 1
Characteristics of patient with samples obtained after
cardiac surgery. Data are shown as median (interquartile
range), n (%), or mean ± SD. Abbreviations are as
follows: AKIN, Acute Kidney Injury Network; CABG, coronary
artery bypass graft; CHF, congestive heart failure.

| | No AKI | AKI |
|---|---|---|
| N | 16 | 16 |
| % female | 21 | 56 |
| % black | 14 | 19 |
| Age (years) | 67 | 68 |
| Wt (kg) | 79 | 83 |
| % CHF | 55 | 13 |
| % Diabetes | 44 | 56 |
| % CABG | 88 | 88 |
| % valve | 44 | 19 |
| % Bypass | 86 | 75 |
| Bypass time (min) | 112 | 115 |
| Baseline | 1.4 | 1.3 |
| Creatinine (mg/dl) | | |
| Collection | 1.4 | 2.7 |
| Creatinine (mg/dl) | | |
| Time to collection | 1.5 | 1.5 |
| (days) | | |
| % Mortality | 0 | 25 |
| Days to Discharge | 10 | 19 |
| or Death | | |

### CONCLUSIONS

Studies presented here utilized a mouse model of FR-induced AKI and provide evidence for the first time that urinary ATPSβ is increased in mice subjected to I/R-induced AKI and this increase correlates with mitochondrial disruption in the kidneys of these mice. Analysis of human clinical samples further showed that urinary ATPSβ protein was elevated in patients who developed severe AKI after cardiac surgery when compared to subjects who did not develop AKI after cardiac surgery. These results provide evidence that increases in urinary ATPSβ protein could serve as a clinical biomarker of renal mitochondrial dysfunction in post-operative AKI and may enable novel therapies for AKI.

Data presented here indicate that full-length urinary ATPSβ protein (but not cleaved ATPSβ) was significantly elevated in mice subjected to 10 and 15 min I/R-AKI when compared to sham and 5 min I/R mice (FIGS. 4D-F). Increase in urinary ATPSβ in 15 min I/R could be a result of necrosis and sloughing off of epithelial cells into tubular lumen due to exaggerated renal injury (Bonventre et al., 2010 and Bonventre et al., 2003). This is consistent with increased urinary NGAL (FIG. 4C), a sensitive and specific biomarker of AKI which was shown to be up-regulated in tubular epithelial cells during the course of I/R injury and excreted into the urine after injury along with dead and denuded epithelial cells (Charlton et al., 2014). Interestingly, full-length urinary ATPSβ was also significantly elevated in 10 min I/R mice in the absence of evident renal dysfunction or damage (FIGS. 4A-C; FIG. 8). ATPSβ is a 52-56 kDa protein which is below the cutoff for glomerular filtration of approximately 60 kDa (Meibohm et al., 2012). The possibility that 10 and 15 min I/R-induced AKI might have some non-renal mitochondrial effects and leakage of ATPSβ into the serum can be ruled out because urinary ATPSβ is elevated similarly in 10 and 15 min I/R mice in spite of a difference in serum creatinine (FIG. 4B; FIG. 8). These results suggest that sub-lethal damage to mitochondria in the absence of necrotic cell death might have induced MPTP and led to translocation of ATPSβ into the cytosol. It has been demonstrated that superoxide can escape from the intermembrane space through voltage-dependent anion channel located in the mitochondrial outer membrane after sub-lethal cellular damage during AKI (Che et al., 2014). Similar mechanisms might exist for ATPSβ once mitochondrial disruption is initiated. However, the mechanisms of its expulsion into the tubular lumen and urine are not clear.

Furthermore, the studies herein show that renal cortical nuclear-encoded mitochondrial ATPSβ decreased in 15 min I/R group (FIGS. 5A-B) suggesting FR-induced mitochondrial disruption in AKI. The loss of mitochondrial proteins would result in disruption of mitochondrial function as previously demonstrated (Nath et al., 1998). It has also recently been demonstrated that renal mitochondrial dysfunction in I/R-AKI mice is persistent until 6 days after I/R-AKI and elevation in serum creatinine concomitant with continual suppression of mitochondrial- and nuclear-encoded genes and proteins of the electron transport chain (ETC) and mitochondrial function (Funk et al., 2012; Jesinkey et al., 2014). Studies herein indicate that persistent elevation in urinary ATPSβ until 72 h after I/R are consistent with this report and would further suggest that respiratory disruption persists over a time course after AKI (FIG. 6). Thus, urinary ATPSβ is not only an early indicator of renal mitochondrial dysfunction but its elevations in urine may also predict persistent mitochondrial dysfunction during the course of AKI.

The human data provided here also indicates that increased urinary ATPSβ in patients with severe AKI predicts renal mitochondrial dysfunction when compared to no AKI subjects. Comparing the changes in ATPSβ protein to the amount of injury, which occurred as measured by the magnitude of change in serum creatinine and the patient's outcome, will gain a better understanding of the changes that occur to mitochondria during AKI. Non-renal effects like bypass time, coexisting disease (diabetes) and cardiovascular effects (CABG) were similar between no AKI and AKI patient populations suggesting specificity for urinary ATPSβ in this study (see, Table 1). Also, fewer AKI patients with elevated urinary ATPSβ have CHF and valve replacements suggesting that elevated ATPSβ may be a sensitive and specific indicator of renal mitochondrial disruption in AKI. Another important finding in the studies shown here is that both mice and human AKI urine samples have increased VVDLLAPYAK (SEQ ID NO: 1) peptide that corresponds to N-terminus region of ATPSβ (FIG. 7E).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Alge et al., Urinary angiotensinogen and risk of severe AKI. Clin J Am Soc Nephrol, 8: 184-193, 2013.
Ball et al., Identification of the major site of O-linked beta-N-acetylglucosamine modification in the C terminus of insulin receptor substrate-1. Mol Cell Proteomics, 5: 313-323, 2006.
Bonventre, Pathophysiology of AKI: injury and normal and abnormal repair. Contributions to nephrology, 165: 9-17, 2010.
Bonventre et al., Recent advances in the pathophysiology of ischemic acute renal failure. Journal of the American Society of Nephrology: JASN, 14: 2199-2210, 2003.
Charlton et al., A basic science view of acute kidney injury biomarkers. Nephrology, dialysis, transplantation: official publication of the European Dialysis and Transplant Association—European Renal Association, 2014.
Che et al., Mitochondrial dysfunction in the pathophysiology of renal diseases. Am J Physiol Renal Physiol, 306: F367-378, 2014.
Coen et al., NMR-based metabolic profiling and metabonomic approaches to problems in molecular toxicology. Chem Res Toxicol 21, 9 (January, 2008).
Funk et al., Persistent disruption of mitochondrial homeostasis after acute kidney injury. American journal of physiology Renal physiology, 302: F853-864, 2012.
Hall et al., The not so ‘mighty chondrion’: emergence of renal diseases due to mitochondrial dysfunction. Nephron Physiol 105, p1 (2007).
Hoste et al., RIFLE criteria for acute kidney injury are associated with hospital mortality in critically ill patients: a cohort analysis. Crit Care 2006 May 12; 10(3):R73.
Jesinkey et al., Formoterol Restores Mitochondrial and Renal Function after Ischemia-Reperfusion Injury. Journal of the American Society of Nephrology: JASN, 2014.
Kim et al., N-acetylcysteine attenuates glycerol-induced acute kidney injury by regulating MAPKs and Bcl-2 family proteins. Nephrol Dial Transplant 25, 1435 (May, 2010).
Korrapati et al., Suramin: a potential therapy for diabetic nephropathy. PLoS One, 8: e73655, 2013.
Korrapati et al., Diabetes-induced renal injury in rats is attenuated by suramin. The Journal of pharmacology and experimental therapeutics, 343: 34-43, 2012.
Leonard et al., VEGF-121 preserves renal microvessel structure and ameliorates secondary renal disease following acute kidney injury. Am J Physiol Renal Physiol 295, F1648 (December, 2008).
Mangano et al., Renal dysfunction after myocardial revascularization: risk factors, adverse outcomes, and hospital resource utilization. The Multicenter Study of Perioperative Ischemia Research Group. Ann Intern Med 1998 Feb. 1; 128(3):194-203.
Meibohm et al., Characterizing the impact of renal impairment on the clinical pharmacology of biologics. Journal of clinical pharmacology, 52: 54S-628, 2012.
Nath et al., Intracellular targets in heme protein-induced renal injury. Kidney international, 53: 100-111, 1998.
Satoh et al., A novel free radical scavenger, edarabone, protects against cisplatin-induced acute renal damage in vitro and in vivo. J. Pharmacol. Eptl. Ther. 305:1183-90, 2003.
Satoh et al., Mitochondrial damage-induced impairment of angiogenesis in the aging rat kidney. Lab. Invest. 91:190-202, 2011.
Schnellmann et al., Pentachlorobutadienyl-L-cysteine uncouples oxidative phosphorylation by dissipating the proton gradient. Toxicol Appl Pharmacol 100, 498, 1989.
Sharma et al., Diabetic kidney disease in the db/db mouse. Am J Physiol Renal Physiol., 284(6):F1138-44, 2003.
Wenz et al., Activation of the PPAR/PGC-1alpha pathway prevents a bioenergetic deficit and effectively improves a mitochondrial myopathy phenotype. Cell Metab. 2008 8(3):249-5.
Zager et al., The influence of mannitol on myoglobinuric acute renal failure: functional, biochemical, and morphological assessments. J Am Soc Nephrol 2, 848 (October, 1991).
Zager, Mitochondrial free radical production induces lipid peroxidation during myohemoglobinuria. Kidney Int 49, 741 (March, 1996).
Zhuang et al., Suramin promotes recovery from renal ischemia/reperfusion injury in mice. Kidney Int 75, 304 (February, 2009).

1. An assay method for assessing mitochondrial dysfunction comprising selectively measuring a level of triphosphate synthase (ATPS) β protein in a blood or urine sample from a subject.

2. The assay method of claim 1, wherein the subject has or is suspected of having kidney dysfunction.

3. The assay method of claim 2, wherein the kidney dysfunction is a kidney injury, chronic kidney disease or diabetic nephropathy.

4. The assay method of claim 1, wherein the subject has undergone cardiac surgery.

5. The assay method of claim 4, wherein the subject was put on bypass during the cardiac surgery.

6. The assay method of claim 1, wherein the subject has diabetes.

7. The assay method of claim 1, wherein the subject is a human.

8. The assay method of claim 1, wherein the sample is a urine sample.

9. The assay method of claim 1, wherein measuring the level of ATPSβ comprises measuring a level of an ATPSβ cleavage product.

10. The assay method of claim 9, wherein the ATPSβ cleavage product has a mass of about 20-25 kDa or has a sequence of SEQ ID NO: 1, 4, 5, 6, 7, 8 or 9.

11. (canceled)

12. The assay method of claim 1, wherein measuring the level of an ATPSβ comprises performing an immunological assay.

13. The assay method of claim 12, wherein the immunological assay is an ELISA assay.

14. The assay method of claim 1, wherein measuring the level of the ATPSβ comprises performing mass spectroscopy.

15. The assay method of claim 14, wherein the mass spectrometry further comprises multiple reaction monitoring.

16. The assay method of claim 1, wherein measuring the level of an ATPSβ comprises normalizing the measured level to a reference.

17. The assay of claim 16, wherein the reference is the total protein level in the sample.

18. The assay of claim 16, wherein the reference is the level of another polypeptide in the sample.

19. The assay of claim 16, wherein the reference is the level of creatinine in the sample.

20. The assay of claim 1, further comprising measuring the level of creatinine in the sample.

21. An isolated immunological complex comprising an ATPSβ cleavage product and an ATPSβ-binding antibody.

22-31. (canceled)

32. An isolated antibody that binds immunologically to a peptide consisting of the sequence of SEQ ID NO: 1, 4, 5, 6, 7, 8 or 9.

33-44. (canceled)


SEQUENCE LISTING (9 sequences)

SEQ ID NO: 1 - protein, 10 aa, Homo sapiens
VVDLLAPYAK

SEQ ID NO: 2 - protein, 529 aa, Homo sapiens
MLGFVGRVAAAPASGALRRLTPSASLPPAQLLLRAAPTAVHPVRDYAAQTSPSPKAGAATGRIVAVIGAVVDVQFDEGLPPILNALEVQGRETRLVLEVAQHLGESTVRTIAMDGTEGLVRGQKVLDSGAPIKIPVGPETLGRIMNVIGEPIDERGPIKTKQFAPIHAEAPEFMEMSVEQEILVTGIKVVDLLAPYAKGGKIGLFGGAGVGKTVLIMELINNVAKAHGGYSVFAGVGERTREGNDLYHEMIESGVINLKDATSKVALVYGQMNEPPGARARVALTGLTVAEYFRDQEGQDVLLFIDNIFRFTQAGSEVSALLGRIPSAVGYQPTLATDMGTMQERITTTKKGSITSVQAIYVPADDLTDPAPATTFAHLDATTVLSRAIAELGIYPAVDPLDSTSRIMDPNIVGSEHYDVARGVQKILQDYKSLQDIIAILGMDELSEEDKLTVSRARKIQRFLSQPFQVAEVFTGHMGKLVPLKETIKGFQQILAGEYDHLPEQAFYMVGPIEEAVAKADKLAEEHSS

SEQ ID NO: 3 - protein, 529 aa, Rattus norvegicus
MLSLVGRVASASASGALRGLNPLAALPQAHLLLRTAPAGVHPARDYAAQSSAAPKAGTATGQIVAVIGAVVDVQFDEGLPPILNALEVQGRESRLVLEVAQHLGESTVRTIAMDGTEGLVRGQKVLDSGAPIKIPVGPETLGRIMNVIGEPIDERGPIKTKQFAPIHAEAPEFIEMSVEQEILVTGIKVVDLLAPYAKGGKIGLFGGAGVGKTVLIMELINNVAKAHGGYSVFAGVGERTREGNDLYHEMIESGVINLKDATSKVALVYGQMNEPPGARARVALTGLTVAEYFRDQEGQDVLLFIDNIFRFTQAGSEVSALLGRIPSAVGYQPTLATDMGTMQERITTTKKGSITSVQAIYVPADDLTDPAPATTFAHLDATTVLSRAIAELGIYPAVDPLDSTSRIMDPNIVGSEHYDVARGVQKILQDYKSLQDIIAILGMDELSEEDKLTVSRARKIQRFLSQPFQVAEVFTGHMGKLVPLKETIKGFQQILAGDYDHLPEQAFYMVGPIEEAVAKADKLAEEHGS

SEQ ID NO: 4 - protein, 15 aa, Homo sapiens
LVLEVAQHLGESTVR

SEQ ID NO: 5 - protein, 31 aa, Homo sapiens
VLDSGAPIKIPVGPETLGRIMNVIGEPIDER

SEQ ID NO: 6 - protein, 11 aa, Homo sapiens
IGLFGGAGVGK

SEQ ID NO: 7 - protein, 15 aa, Homo sapiens
VALVYGQMNEPPGAR

SEQ ID NO: 8 - protein, 43 aa, Homo sapiens
VALTGLTVAEYFRDQEGQDVLLFIDNIFRFTQAGSEVSALLGR

SEQ ID NO: 9 - protein, 35 aa, Homo sapiens
AIAELGIYPAVDPLDSTSRIMDPNIVGSEHYDVAR